(19) United States
(12) Patent Application Publication
(10) Pub. No.: US 2024/0278026 A1
(43) Pub. Date: Aug. 22, 2024

(54) LEAD INSERTION VERIFICATION

(71) Applicant: Cardiac Pacemakers, Inc., St. Paul, MN (US)

(72) Inventors: Moira B. Sweeney, St. Paul, MN (US); James Michael English, Cahir (IE); Jamie Murphy, Abbeyfeale (IE); Robert Allen Jones, Lake Elmo, MN (US); Trey Henry Achterhoff, Arden Hills, MN (US)

(21) Appl. No.: 18/581,309

(22) Filed: Feb. 19, 2024

Related U.S. Application Data
(60) Provisional application No. 63/447,451, filed on Feb. 22, 2023.

(51) Int. Cl. A61N 1/375; H01R 13/405; H01R 13/641; H01R 43/20

(52) U.S. Cl. CPC A61N 1/3752; H01R 13/641; H01R 43/20; H01R 13/405; H01R 2201/12

(57) ABSTRACT

A connector apparatus for a medical device includes connector apparatus of a medical device to provide electrical contact to at least one conductive lead, the apparatus comprising: at least one lead receptacle including an opening to receive the at least one conductive lead, wherein the at least one lead receptacle includes: at least a first electrode to contact the at least one conductive lead when the conductive lead is inserted into the lead receptacle; and a moveable visual indicator to be moved to become visible through the connector apparatus when the at least one conductive lead is inserted in the lead receptacle and contacts the at least one electrode.

### CLAIM OF PRIORITY

This application claims the benefit of U.S. Provisional Application No. 63/447,451 filed on Feb. 22, 2023, which is hereby incorporated by reference in its entirety.

### TECHNICAL FIELD

This patent application relates to implantable medical devices and, in particular, a device connector for implantable leads.

### BACKGROUND

Implantable medical devices or partially implantable medical devices can include a hermetically sealed metal case that houses electronic circuits. For some implantable or partially implantable cardiac rhythm management devices, electrically conductive implantable leads are used to sense electrical signals in the body of a patient, and to deliver electrical signals to the body as part of electrical therapy. Typically, the leads are implanted and then connected to the implantable or partially implantable medical device. For comfort and satisfaction of the patient it is desired for the connector to be robust, while for a physician it is desired for the connector to be easy to use.

### SUMMARY

Systems and methods are disclosed to lead connector assemblies for medical devices. Example 1 includes subject matter (such as a connector apparatus of a medical device) including at least one lead receptacle including an opening to receive the at least one conductive lead. The lead receptacle includes at least a first electrode to contact the at least one conductive lead when the conductive lead is inserted into the lead receptacle, and a moveable visual indicator that moves to become visible through the connector apparatus when a conductive lead is properly inserted in the lead receptacle.

In Example 2, the subject matter of Example 1 optionally includes a visual indicator having a ring shape including an outer diameter and an inner diameter, and inner diameter is sized to receive an end of the at least one conductive lead and the visual indicator is to slide further into the lead receptacle when the at least one conductive lead is inserted into the lead receptacle.

In Example 3, the subject matter of one or both of Examples 1 and 2 optionally includes a visual indicator to move from a non-visible position in the lead receptacle to a visible position in the lead receptacle when the at least one conductive lead is inserted in the lead receptacle to an end of the lead receptacle.

In Example 4, the subject matter of one or any combination of Examples 1-3 optionally includes a lead receptacle that includes a cylindrical opening that includes a non-conductive material, and the visual indicator includes a cylindrical shape and includes silicone.

In Example 5, the subject matter of one or any combination of Examples 1-4 optionally includes a visual indicator that includes a color visible through an end of the lead receptacle when the at least one conductive lead contacts the at least one electrode.

In Example 6, the subject matter of one or any combination of Examples 1-5 optionally includes a lead receptacle that includes a clear end cap, and the visual indicator includes a color visible through the clear end cap when the at least one conductive lead is inserted in the lead receptacle and contacts the at least one electrode.

In Example 7, the subject matter of one or any combination of Examples 1-6 optionally includes a header connector for the medical device, and the header connector includes a mold layer covering the lead receptacle and the visual indicator is moveable to be visible through the mold layer when the at least one conductive lead is inserted in the lead receptacle and contacts the at least one electrode.

In Example 8, the subject matter of one or any combination of Examples 1-7 optionally includes at least one lead receptacle including a ridge to hold the moveable visual indicator in a non-visible position in the lead receptacle until the at least one conductive lead is inserted in the lead receptacle.

Example 9 includes subject matter (such as a method of making a connector assembly for a medical device) or can optionally be combined with one or any combination of Examples 1-8 to include such subject matter, including forming a lead receptacle to receive a conductive lead. Forming the lead receptacle includes forming a cylindrical tube having a bore to receive an end of the conductive lead and forming at least one electrode in the bore to contact the conductive lead when the conductive lead is inserted into the bore. The method further includes arranging a moveable visual indicator within the bore in a first position with the visual indicator not visible external to the lead receptacle. The visual indicator is moveable to a second position in the bore with the visual indicator visible external to the lead receptacle when the end of the conductive lead is properly inserted in the bore.

In Example 10, the subject matter of Example 9 optionally includes forming multiple lead receptacles, each lead receptacle including at least one electrode and a visual indicator; attaching conductive wires to the electrodes of the multiple lead receptacles; and forming a mold layer covering the lead receptacles and conductive wires, and wherein the visual indicators are visible within the mold layer when the visual indicators are in the second position in the bore.

In Example 11, the subject matter of one or both of Examples 9 and 10 optionally includes arranging a visual indicator having a ring shape within the bore, wherein an inner diameter of the ring-shaped visual indicator is sized to receive an end of the conductive lead.

In Example 12, the subject matter of one or any combination of Examples 9-11 optionally includes forming a ridge in the bore to hold the visual indicator at the first position in the bore.

In Example 13, the subject matter of one or any combination of Examples 9-12 optionally includes a cylindrical tube having a clear end, and arranging the visual indicator next to the at least one electrode in the first position, and the visual indicator is moveable to the second position at the clear end of the cylindrical tube when the conductive lead is inserted in the bore.

Example 14 includes subject matter (such as a header connector assembly for a medical device) or can optionally be combined with one or nay combination of Examples 1-13 to include such subject matter, including multiple lead receptacles and a header block. Each lead receptacle includes a cylindrical tube having a bore to receive an end of a conductive lead; at least one electrode to contact the conductive lead when the conductive lead is inserted into the bore; and a moveable visual indicator. The header block includes a mold layer. The multiple lead receptacles are arranged within the mold layer and the header block further includes multiple conductive wires to contact the electrodes of the lead receptacles; and the moveable visual indicator of a lead receptacle is configured to move from a non-visible position in the header block to a visible position in the header block when the conductive lead is inserted in the lead receptacle and contacts the at least one electrode.

In Example 15, the subject matter of Example 14, optionally includes the moveable visual indicator of each lead receptacle has a ring shape having an inner diameter that is sized to receive an end of the conductive lead, and the moveable visual indicator is to slide further into the lead receptacle when the conductive lead is inserted into the lead receptacle.

In Example 16, the subject matter of one or both of Examples 14 and 15 optionally includes the moveable visual indicator of each lead receptacle configured to move from the non-visible position in the header connector to the visible position in the header connector when the conductive lead is inserted in the lead receptacle to an end of the lead receptacle.

In Example 17, the subject matter of one or any combination of Examples 14-16 optionally includes the cylindrical tubes of the lead receptacles including a non-conductive material, and the visual indicator of each lead receptacle including silicone and having a cylindrical shape.

In Example 18, the subject matter of one or any combination of Examples 14-17 optionally includes the visual indicator of each lead receptacle being visible through an end of the lead receptacle when the conductive lead is inserted to contact the at least one electrode of the lead receptacle.

In Example 19, the subject matter of one or any combination of Examples 14-18 optionally includes each lead receptacle including a clear end cap, and the visual indicator of each lead receptacle is to become visible through the clear end cap when the conductive lead is inserted in the lead receptacle and contacts the at least one electrode.

In Example 20, the subject matter of one or any combination of Examples 14-19 optionally includes each lead receptacle including a ridge within the bore to hold the moveable visual indicator in the non-visible position in the header block until the conductive lead is inserted in the lead receptacle.

The non-limiting Examples can be combined in any permutation or combination. This summary is intended to provide an overview of the subject matter of the present application. It is not intended to provide an exclusive or exhaustive explanation of the invention. The detailed description is included to provide further information about the subject matter of the present patent application.

### BRIEF DESCRIPTION OF THE DRAWINGS

FIG. 1 is an illustration of portions of a system that uses an implantable medical device.

FIG. 2 is a view of an example of a medical device.

FIG. 3 is a view of the end portions of an example of lead receptacles of a connector for a medical device.

FIG. 4 is a close-up view of an end portion of an example of a lead receptacle of a connector assembly for a medical device.

FIG. 5 is a view of an example of a header block that includes multiple lead receptacles.

FIG. 6 is a flow diagram of an example of a method of manufacture for a connector assembly for a medical device.

### DETAILED DESCRIPTION

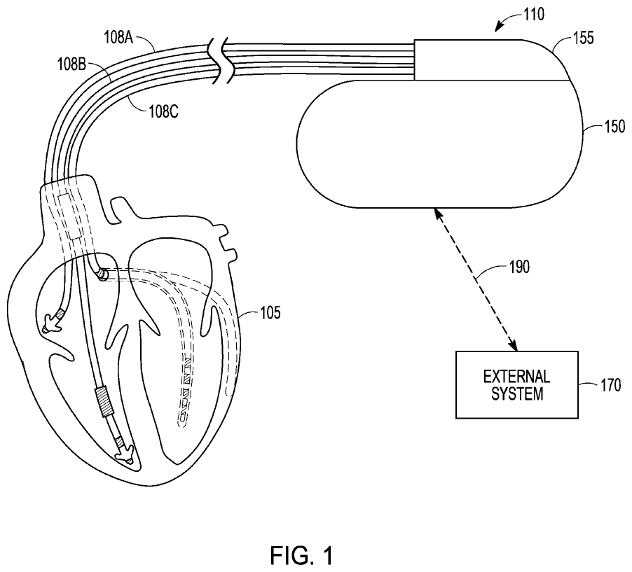

This document relates to a connector assembly for an ambulatory medical device. FIG. 1 is an illustration of portions of a system that uses an implantable medical device (IMD). Some examples of the IMD 110 include a pacemaker, a defibrillator, a cardiac resynchronization therapy (CRT) device, a combination of such devices, or a diagnostic-only device. The system also typically includes an IMD programmer or other external device 170 that communicates wireless signals 190 with the IMD 110, such as by using radio frequency (RF) or other telemetry signals.

The IMD 110 can be coupled by one or more conductive leads 108A-C to heart 105. The cardiac leads 108A-C in the example of FIG. 1 include a proximal end that is coupled to IMD 110 and a distal end, coupled by electrical contacts or “electrodes” to one or more portions of a heart 105. The electrodes typically deliver cardioversion, defibrillation, pacing, or resynchronization therapy, or combinations thereof to at least one chamber of the heart 105. The electrodes may be electrically coupled to sense amplifiers to sense electrical cardiac signals. Sensed electrical cardiac signals can be sampled to create an electrogram. An electrogram can be analyzed by the IMD and/or can be stored in the IMD and later communicated to an external device where the sampled signals can be displayed for analysis.

The cardiac leads 108A-C include right atrial (RA) lead 108A, right ventricle (RV) lead 108B, and a third cardiac lead 108C for placement in a coronary vein lying epicardially on the left ventricle (LV) via the coronary vein.

The IMD 110 includes a hermetically-sealed IMD housing or can 150 that houses electronic circuits and a header connector 155. The cardiac leads 108A-C are connected to the IMD through the header connector 155. For implantable cardiac rhythm management devices, electrical signals sensed in the body and electrical signals delivered to the body need to pass through the hermetic seal. This is accomplished with feedthroughs. Feedthroughs are comprised of an electrical conductor, usually a pin, passing through insulating material and providing connection from circuitry internal to the can to a point external to the can while maintaining the hermetic seal. The header connector 155 provides the electrical connection between conductive leads and the feedthroughs. It is desired for a lead connector of a medical device (such as the header connector 155 in FIG. 1) to provide feedback to the physician regarding proper placement of the lead in a lead connector.

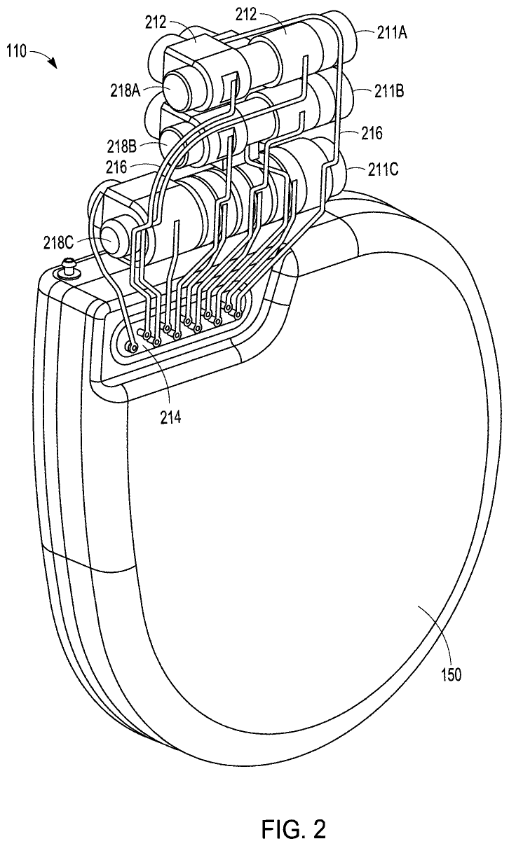

FIG. 2 is a view of another example of a medical device 110. The view shows the metal case 150 of the medical device and shows the header connector without the mold material to show the lead receptacles. The header connector includes three lead receptacles 211A-C into which conductive leads can be inserted so that electrodes of the conductive leads contact electrodes 212 of the lead receptacles 211A-C. The lead receptacles can be configured to receive different types of implantable leads. For example, lead receptacles 211A-B may be designed to receive International Standard One (IS-1) types of leads. Lead receptacle 211C may be designed to receive IS-4 types of leads. FIG. 2 also shows a feedthrough connector assembly 214 of the medical device 110. The feedthroughs are connected to the lead receptacles 211A-C by conductive wires 216.

In general, when implanting the medical device, the conductive leads are placed first, and distal ends of the leads are inserted into the lead receptacles 211A-C after the leads are placed. The leads need to be properly placed in the header connector or optimum operation. When properly placed, the electrodes of the leads match up with the electrodes 212 of the lead receptacles. This proper placement is usually achieved by inserting the leads into the lead receptacles 211A-C until the leads reach the receptacle ends 218A-C. However, it can be difficult to determine if the lead is properly inserted. The lead may feel to the physician that it is completely in before the lead actually reaches the end of the lead receptacle. This can result in the lead not operating properly for the patient.

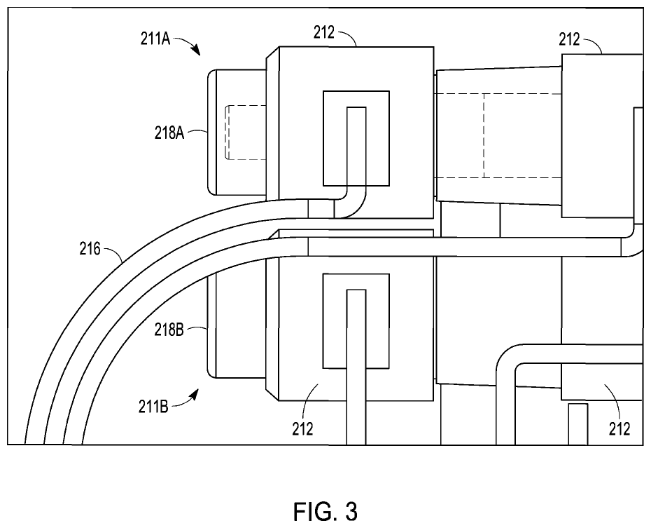

FIG. 3 is a view of the end portions of an example of lead receptacles 211A and 211B. The lead receptacles include a cylindrical tube made of non-conductive material. The cylindrical tube includes a bore, and the conductive leads are inserted into the bore of the lead receptacles 211A, 211B. To provide visual feedback of proper insertion, the lead receptacles 211A, 211B can be clear to allow the physician to see the lead end reach the end of the lead receptacle. However, the shape of the lead receptacles 211A, 211B causes the optical effect of making the lead ends appear smaller than they actually are. This makes it difficult for a physician to visually verify that the lead is properly inserted in the lead receptacle.

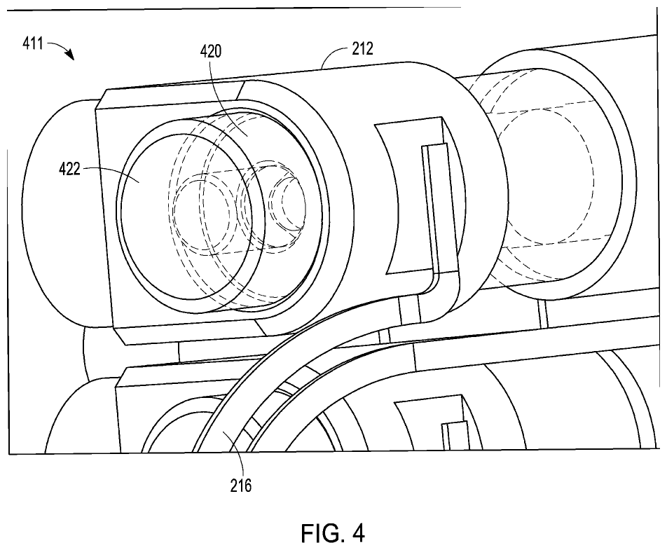

FIG. 4 is a close-up view of an end portion of an example of a lead receptacle 411 of a header connector assembly. The lead receptacle 411 includes an electrode 212 that contacts the lead when the distal end of the lead is properly inserted into the bore of the lead receptacle 411. The lead receptacle 411 includes a moveable visual indicator 420. The visual indicator 420 is slidable within the bore of the lead receptacle 411. In the example of FIG. 4, the visual indicator is shown in a position next to the electrode 212 and away from the end of the lead receptacle 411. When a lead is inserted into the led receptacle, the lead pushes the visual indicator 420 toward the end of the lead receptacle 411. When the visual indicator 420 is at the end of the lead receptacle the lead is properly inserted in the lead receptacle 411. In some examples, the lead is properly inserted in the lead receptacle 411 when the position of the electrode of the lead is adjacent the electrode 212 of the lead receptacle 411. In some examples, the lead is properly inserted in the lead receptacle 411 when the end of the lead reaches the end of the lead receptacle 411.

The lead receptacle 411 has a clear end. In some examples, the lead receptacle 411 has a clear end cap 422. The visual indicator 420 has a color (e.g., blue, red, orange, etc.) that makes it easy to recognize when the visual indicator 420 is at the end of the lead receptacle 411. In some examples, the visual indicator 420 is not visible before the lead is inserted. As shown in the example of FIG. 4, the electrode 212 may be a ring electrode, and the visual indicator is hidden from view by the electrode 212 and is not visible to the physician inserting the lead. When the lead is properly inserted, the colored visual indicator 420 is in a position that is visible to the physician.

In the example of FIG. 4, the visual indicator 420 has a ring shape with an outer diameter and an inner diameter. The inner diameter of the ring can be sized for the lead tip to fit through, and the body of the lead moves the visual indicator.

When the visual indicator 420 is at the end of the lead receptacle, the lead end is at the end of the lead receptacle. In certain examples, the visual indicator has a cylindrical shape, and the center of the cylindrical visual indicator may be solid or hollow. The visual indicator may be made of silicone. The visual indicator 420 is configured by shape and size to add negligible insertion force to the insertion force needed to insert the lead in the lead receptacle 411. The lead receptacle 411 may include a ridge within the bore that holds the visual indicator to keep the visual indicator 420 from sliding out of the bore.

Having the visual indicator 420 included in a part of the header connector removes the need to place any visual assistance device on the lead. If the lead receptacle is sized for an IS-1 lead, the visual indicator 420 provides visual assistance in insertion of any lead complying with the IS-1 standard.

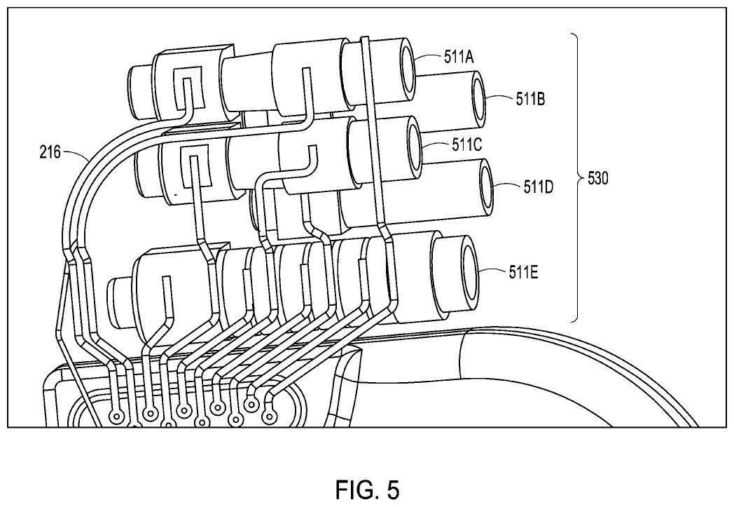

The lead receptacle 411 can be included in a header block that includes multiple lead receptacles. FIG. 5 is a view of an example of a header block 530 that includes five lead receptacles 511A-511E. The lead receptacles of the header block may have different sizes corresponding to different types of leads. For example, the header block may include lead receptacles for any combination of IS-1, IS-4, and DF1 leads, etc. Different sized lead receptacles may include a different sized visual indicator 420. The header block 530 may include a mold layer covering the lead receptacles 511A-511E and connector wires of the header block 530. In certain examples, the mold can include clear epoxy. The visual indicators of the lead receptacles are moveable to be visible through the mold layer when a conductive lead is properly inserted in the lead receptacle.

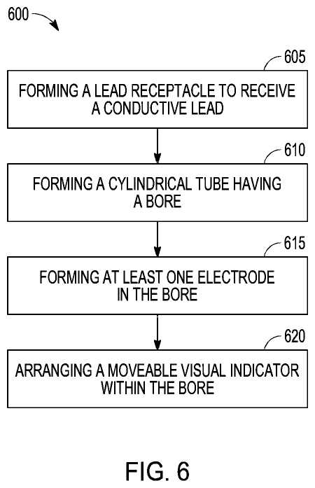

FIG. 6 is a flow diagram of an example of a method 600 of manufacture for a connector assembly for a medical device. At block 605, at least one lead receptacle is formed for the connector assembly, such as the lead receptacle 411 of FIG. 4 for example. Forming the lead receptacle can include forming a cylindrical tube having a bore sized to receive a conductive lead at block 610 and forming at least one electrode in the bore to contact the conductive lead when the conductive lead is inserted into the bore at block 615. Multiple electrodes (e.g., ring electrodes, or segmented electrodes that extend less than all the way around the bore) can be formed for the lead receptacle to match the electrodes of the conductive lead.

At block 620, a moveable visual indicator (e.g., visual indicator 420 of FIG. 4) is arranged within the bore. The visual indicator can be arranged in a first position with the visual indicator not visible external to the lead receptacle. The visual indicator is moveable within the bore to a second position within the bore when the conductive lead is properly inserted in the lead receptacle. The visual indicator is visible external to the lead receptacle when the visual indicator is moved to the second position.

The method 600 can include forming a ring-shaped moveable visual indicator and arranging the ring-shaped visual indicator in the bore. The method 600 can include forming a ridge in the bore to hold the visual indicator at the first position until the conductive lead is inserted. Forming the cylindrical tube can include forming a cylindrical tube having a clear end (e.g., a clear end cap) and the second position is the end of the bore of the cylindrical tube.

The method 600 can include forming multiple lead receptacles for the connector assembly. A moveable visual indicator can be arranged in each of the lead receptacles. The electrodes of the lead receptacles can be connected to conductive wires that can be connected to feedthroughs of the medical device. The lead receptacles and the conductive wires can be encapsulated in a mold layer to form a header connector assembly for the medical device.

The devices described herein provide a connector with visual feedback for connecting electrical leads to a medical device. The visual feedback is provided by moveable visual indicators included in the connector, rather than modifying the leads to provide the feedback. Having the connector provide the visual feedback results in visual feedback being provided to the physician when using and compliant lead made by any manufacturer. The visual indicators add negligible insertion force, thereby maintaining low insertion force for a low insertion force lead connection.

### Additional Description

The above detailed description includes references to the accompanying drawings, which form a part of the detailed description. The drawings show, by way of illustration, specific embodiments in which the invention can be practiced. These embodiments are also referred to herein as “examples.” All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times. These computer-readable media can include, but are not limited to, hard disks, removable magnetic disks, removable optical disks (e.g., compact disks and digital video disks), magnetic cassettes, memory cards or sticks, random access memories (RAM's), read only memories (ROM's), and the like. In some examples, a carrier medium can carry code implementing the methods. The term “carrier medium” can be used to represent carrier waves on which code is transmitted.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. A connector apparatus of a medical device to provide electrical contact to at least one conductive lead, the apparatus comprising:
at least one lead receptacle including an opening to receive the at least one conductive lead, wherein the at least one lead receptacle includes:
at least a first electrode to contact the at least one conductive lead when the conductive lead is inserted into the lead receptacle; and
a moveable visual indicator to be moved to become visible through the connector apparatus when the at least one conductive lead is inserted in the lead receptacle and contacts the at least one electrode.

2. The apparatus of claim 1, wherein the visual indicator has a ring shape including an outer diameter and an inner diameter, and inner diameter is sized to receive an end of the at least one conductive lead and the visual indicator is to slide further into the lead receptacle when the at least one conductive lead is inserted into the lead receptacle.

3. The apparatus of claim 1, wherein the visual indicator is to move from a non-visible position in the lead receptacle to a visible position in the lead receptacle when the at least one conductive lead is inserted in the lead receptacle to an end of the lead receptacle.

4. The apparatus of claim 1, wherein lead receptacle includes a cylindrical opening that includes a non-conductive material, and the visual indicator includes a cylindrical shape and includes silicone.

5. The apparatus of claim 1, wherein the visual indicator includes a color visible through an end of the lead receptacle when the at least one conductive lead contacts the at least one electrode.

6. The apparatus of claim 1, wherein the lead receptacle includes a clear end cap, and the visual indicator includes a color visible through the clear end cap when the at least one conductive lead is inserted in the lead receptacle and contacts the at least one electrode.

7. The apparatus of claim 1, wherein the connector apparatus includes a header connector for the medical device, and the header connector includes a mold layer covering the lead receptacle and the visual indicator is moveable to be visible through the mold layer when the at least one conductive lead is inserted in the lead receptacle and contacts the at least one electrode.

8. The apparatus of claim 1, wherein the at least one lead receptacle includes a ridge to hold the moveable visual indicator in a non-visible position in the lead receptacle until the at least one conductive lead is inserted in the lead receptacle.

9. A method of making a connector assembly for a medical device, the method including:
forming a lead receptacle to receive a conductive lead, including:
forming a cylindrical tube having a bore to receive an end of the conductive lead;
forming at least one electrode in the bore to contact the conductive lead when the conductive lead is inserted into the bore; and
arranging a moveable visual indicator within the bore in a first position with the visual indicator not visible external to the lead receptacle, wherein the visual indicator is moveable to a second position in the bore with the visual indicator visible external to the lead receptacle when the end of the conductive lead is inserted in the bore and contacts the at least one electrode.

10. The method of claim 9, including:
forming multiple lead receptacles, each lead receptacle including at least one electrode and a visual indicator;
attaching conductive wires to the electrodes of the multiple lead receptacles; and
forming a mold layer covering the lead receptacles and conductive wires, and wherein the visual indicators are visible within the mold layer when the visual indicators are in the second position in the bore.

11. The method of claim 9, wherein the arranging the visual indicator includes arranging a visual indicator having a ring shape within the bore, wherein an inner diameter of the ring-shaped visual indicator is sized to receive an end of the conductive lead.

12. The method of claim 9, including forming a ridge in the bore to hold the visual indicator at the first position in the bore.

13. The method of claim 9,
wherein the cylindrical tube has a clear end; and
wherein the arranging the visual indicator includes arranging the visual indicator next to the at least one electrode in the first position, and the visual indicator is moveable to the second position at the clear end of the cylindrical tube when the conductive lead is inserted in the bore.

14. A header connector assembly of a medical device, the assembly comprising:
multiple lead receptacles, each lead receptacle including:
a cylindrical tube having a bore to receive an end of a conductive lead;
at least one electrode to contact the conductive lead when the conductive lead is inserted into the bore; and
a moveable visual indicator; and
a header block including a mold layer, wherein the multiple lead receptacles are arranged within the mold layer and the header block further includes multiple conductive wires to contact the electrodes of the lead receptacles; and
wherein the moveable visual indicator of a lead receptacle is configured to move from a non-visible position in the header block to a visible position in the header block when the conductive lead is inserted in the lead receptacle and contacts the at least one electrode.

15. The assembly of claim 14, wherein the moveable visual indicator of each lead receptacle has a ring shape having an inner diameter that is sized to receive an end of the conductive lead, and the moveable visual indicator is to slide further into the lead receptacle when the conductive lead is inserted into the lead receptacle.

16. The assembly of claim 14, wherein the moveable visual indicator of each lead receptacle is to move from the non-visible position in the header connector to the visible position in the header connector when the conductive lead is inserted in the lead receptacle to an end of the lead receptacle.

17. The assembly of claim 14, wherein the cylindrical tubes of the lead receptacles include a non-conductive material, and the visual indicator of each lead receptacle includes silicone and includes a cylindrical shape.

18. The assembly of claim 14, wherein the visual indicator of each lead receptacle is visible through an end of the lead receptacle when the conductive lead is inserted to contact the at least one electrode of the lead receptacle.

19. The assembly of claim 14, wherein each lead receptacle includes a clear end cap, and the visual indicator of each lead receptacle is to become visible through the clear end cap when the conductive lead is inserted in the lead receptacle and contacts the at least one electrode.

20. The assembly of claim 14, wherein each lead receptacle includes a ridge within the bore to hold the moveable visual indicator in the non-visible position in the header block until the conductive lead is inserted in the lead receptacle.
